SEQUENCE LISTING (11 sequences)

SEQ ID NO: 1 - DNA, 8 bp, Artificial
  This is a modified Kozak′s sequence
ccgccatg

SEQ ID NO: 2 - DNA, 6 bp, Artificial
  This is a recombinant Eco R1 sequence.
gaattc

SEQ ID NO: 3 - DNA, 713 bp, Porcine circovirus
cagctatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtggagaaggaaaaatggcatcttcaacacccgcctctcccgcaccttcggatatactgtgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttaaaccctaaatgaat

SEQ ID NO: 4 - DNA, 713 bp, Porcine circovirus
ccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattc

SEQ ID NO: 5 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLKP

SEQ ID NO: 6 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 7 - DNA, 756 bp, Artificial
  This sequence is from porcine circovirus type 2, open reading frame 2, together with a portion from the pGEM T-easy vector.
gcggccgcgggaattcgatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattctatcactagtgaattcgcggccgc

SEQ ID NO: 8 - DNA, 10387 bp, Artificial
  This is the porcine circovirus type 2, ORF2 construct, which includes baculovirus and pGEM T-easy coding sequences.
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatcagatctgcagcggccgcgggaattcgatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattctatcactagtgaattcgcggccgccggccgctccagaattctagaaggtacccgggatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcgccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaacgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 9 - protein, 20 aa, Porcine circovirus
SYPRRRYRRRRHHPPSHLGQ

SEQ ID NO: 10 - protein, 19 aa, Porcine circovirus
PRHHYRPRRKNGIFNTTLS

SEQ ID NO: 11 - protein, 233 aa, Artificial
  This is an amino acid sequence for porcine circovirus type 2, open reading frame 2.
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIKKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSIYDQDYNIRVTMYVQFREFNLKDPPLKP

(19) United States
(12) Patent Application Publication
(10) Pub. No.: US 2011/0033495 A1
(43) Pub. Date: Feb. 10, 2011

(54) METHODS AND COMPOSITIONS FOR REDUCING THE IMPACT OF ENTERIC DISEASES

(75) Inventors: Michael Roof, Ames, IA (US); Marc Eichmeyer, Ames, IA (US)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM VETMEDICA, INC., St. Joseph, MO (US)

(21) Appl. No.: 12/867,622

(22) PCT Filed: Feb. 14, 2009
(86) PCT No.: PCT/US09/34164

§ 371 Date: Oct. 6, 2010

Related U.S. Application Data
(60) Provisional application No. 61/029,240, filed on Feb. 15, 2008.

(51) Int. Cl. A61K 39/295; A61K 39/12; A61P 31/04; A61P 31/20

(52) U.S. Cl. 424/201.1; 424/204.1

(57) ABSTRACT

The present invention relates to the use of an immunogenic composition that comprises a PCV2 antigen for treatment of several clinical manifestations (diseases). Preferably, the clinical manifestations are associated with an enteric infection, even more preferably, with enteric disease. The use relates to a method comprising the steps of administering the composition to an animal in need thereof, preferably prior to disease exposure. Administration of PCV2 antigen, preferably ORF2 of PCV2, lessens the incidence and reduces the severity of the enteric disease.

### SEQUENCE LISTING

This application contains a sequence listing in computer readable format, the teachings and content of which are hereby incorporated by reference. The sequence listing is identical with that incorporated in WO06/072065, the teachings and content of which is also incorporated by reference herein.

### BACKGROUND OF THE INVENTION

1. Field of the Invention

The present invention relates to enteric diseases, and methods of reducing the same. More particularly, the present invention relates to methods for reducing the incidence of and/or the severity of enteric diseases. Still more particularly, the present invention relates to methods for reducing the incidence of and/or the severity of enteric diseases by administering to an animal in need thereof an immunogenic composition against a disease not previously associated with an enteric effect. Even more particularly, the present invention relates to vaccinating swine against porcine circovirus, and preferably porcine circovirus type 2. Even more particularly, the present invention relates to vaccinating swine with immunogenic compositions against porcine circovirus type 2 and at least one other pathogen associated with enteric diseases.

2. Description of the Prior Art

Porcine circovirus type 2 (PCV2) is a small (17-22 nm in diameter), icosahedral, non-enveloped DNA virus, which contains a single-stranded circular genome. PCV2 shares approximately 80% sequence identity with porcine circovirus type 1 (PCV1). However, in contrast with PCV1, which is generally non-virulent, swine infected with PCV2 exhibit a syndrome commonly referred to as Post-weaning Multisystemic Wasting Syndrome (PMWS). PMWS is clinically characterized by wasting, paleness of the skin, unthriftiness, respiratory distress, diarrhea, icterus, and jaundice. In some affected swine, a combination of all symptoms will be apparent while other affected swine will only have one or two of these symptoms. During necropsy, microscopic and macroscopic lesions also appear on multiple tissues and organs, with lymphoid organs being the most common site for lesions. A strong correlation has been observed between the amount of PCV2 nucleic acid or antigen and the severity of microscopic lymphoid lesions. Mortality rates for swine infected with PCV2 can approach 80%.

Current approaches to treat PCV2 infections include DNA-based vaccines, such as those described in U.S. Pat. No. 6,703,023. In WO 03/049703 production of a live chimeric vaccine is described, comprising a PCV1 backbone in which an immunogenic gene of a pathogenic PCV2 strains replaces a gene of the PCV1 backbone. WO 99/18214 has provided several PCV2 strains and procedures for the preparation of a killed PCV2 vaccine. An effective ORF-2 based subunit vaccine has been reported in WO 06/072065. Those vaccines described in the prior art were focused solely on the prevention of PCV2 infections in swine, but did not consider any effect on enteric diseases that were caused by PCV2 or on enteric diseases that were caused by PCV2 in combination with another enteric pathogen. Furthermore, the possibility of reducing or eliminating any synergistic effect between PCV2 and other enteric disease-inducing pathogens by administering PCV2 immunogenic compositions was not taught or suggested.

It has been recently discovered that many cases of enteric diseases thought to be caused by organisms other than PCV2 are in fact caused, at in least in part, by PCV2. In other words, animals exhibiting clinical signs or symptoms of infection by an enteric disease causing pathogen were often assumed to be infected with an enteric disease producing organism such as Lawsonia intracellularis, Salmonella, E. coli, Clostridia, or the like. However, actual diagnosis of these animals have shown that many are infected with just PCV2 and others are infected by PCV2 in combination with some other enteric disease inducing pathogen. However, it was never suggested or taught that vaccination against PCV2 would reduce the incidence of and/or severity of enteric disease clinical signs or symptoms.

Accordingly, what is needed in the art is/are immunogenic composition(s) for reducing the incidence of and/or severity of enteric disease caused by PCV2, or PCV2 in combination with another enteric disease causing pathogen.

### SUMMARY OF THE INVENTION

The present invention overcomes the problems inherent in the prior art and provides a distinct advance in the state of the art. The present invention provides methods and immunogenic composition(s) for reducing the incidence of and/or severity of enteric disease caused by PCV2, or PCV2 in combination with another enteric disease causing pathogen. Unless defined otherwise, all technical and scientific terms used herein have the same meanings as commonly understood by one of ordinary skill in the art to which this invention belongs.

The invention takes advantage of the unexpected benefit provided by immunogenic compositions against PCV2, wherein enteric diseases and clinical signs or symptoms associated with those diseases are reduced in incidence and/or severity by administration of the PCV2 immunogenic compositions. This benefit is even more unexpected because the PCV2 immunogenic composition can be, and preferably is administered via an injection, yet still provides the beneficial impact described above. In order to reduce the incidence or severity of enteric disease clinical signs or symptoms, the present invention demonstrates that administering an immunogenic composition against PCV2 or vaccinating against PCV2, preferably prior to PCV2 infection, effectively reduces the incidence of and/or severity of the clinical signs or symptoms of enteric disease. In addition to reducing the incidence of and/or severity of enteric disease clinical signs or symptoms, the incidence and severity of PCV2 is also reduced, thereby contributing to improving the overall health of swine having such vaccination(s). Furthermore, the incidence of and/or severity of enteric disease clinical signs or symptoms caused by PCV2 in combination with another enteric disease causing organism are reduced when vaccinating against PCV2. This effect can be increased when the other disease causing organism is also vaccinated against.

It was surprisingly found that those animals infected with an enteric pathogen and PCV2 exhibited a significant increase in clinical symptoms of enteric disease. In Salmonella/PCV2 infections, lung lesions were enhanced at least 33% in comparison to those animals infected with Salmonella alone. Lung lesions were enhanced at least 13% in animals infected with Lawsonia/PCV2 compared to those infected with Lawsonia alone.

The PCV2 immunogenic composition, as used herein, for example, refers to Ingelvac CircoFLEX®, (Boehringer Ingelheim Vetmedica Inc, St Joseph, Mo., USA), CircoVac® (Merial SAS, Lyon, France), CircoVent (Intervet Inc., Millsboro, Del., USA), or Suvaxyn PCV2 One Dose® (Fort Dodge Animal Health, Kansas City, Kans., USA). The most preferred PCV2 antigen, as used herein, is Ingelvac CircoFLEX®, (Boehringer Ingelheim Vetmedica Inc, St Joseph, Mo., USA).

Enteric pathogens include, but are not limited to: Salmonella spp. (1), in particular S. typhimurium (1a), S. choleraesuis (1b); Astroviruses (2); Rotavirus (3); Transmissible gastroenteritis virus (4); Brachyspira spp (5)., in particular B. hyodysenteriae (5a), B. pilosicoli (5b); Clostridium spp. (6), in particular C. difficile (6a), C. perfringens types A, B and C (6b), C. novyi (6c), C. septicum (6d), C. tetani (6e); Porcine enteric picornaviruses (7); Porcine enteric caliciviruses (8); Lawsonia intracellularis; (9) coronavirus; (10) E. coli; and (11) Lawsonia intracellularis.

L. intracellularis, the causative agent of porcine proliferative enteropathy (“PPE”), affects virtually all animals, including: rabbits, ferrets, hamsters, fox, horses, and other animals as diverse as ostriches and emus. PPE is a common diarrheal disease of growing-finishing and young breeding pigs characterized by hyperplasia and inflammation of the ileum and colon. It often is mild and self-limiting but sometimes causes persistent diarrhea, severe necrotic enteritis, or hemorrhagic enteritis with high mortality.

The bacteria associated with PPE have been referred to as “Campylobacter-like organisms.” S. McOrist et al., Vet. Pathol., Vol. 26, 260-264 (1989). Subsequently, the causative bacteria have been identified as a novel taxonomic genus and species, vernacularly referred to as Ileal symbiont (IS) intracellularis. C. Gebhart et al., Int'l. J. of Systemic Bacteriology, Vol. 43, No. 3, 533-538 (1993). More recently, these novel bacteria have been given the taxonomic name Lawsonia (L.) intracellularis. S. McOrist et al., Int'l. J. of Systemic Bacteriology, Vol. 45, No. 4, 820-825 (1995). These three names have been used interchangeably to refer to the same organism as further identified and described herein. Koch's postulates have been fulfilled by inoculation of pure cultures of L intracellularis into conventionally reared pigs; typical lesions of the disease were produced, and L intracellularis was reisolated from the lesions. The more common, nonhemorrhagic form of the disease often affects 18- to 36-kg pigs and is characterized by sudden onset of diarrhea. The feces are watery to pasty, brownish, or faintly blood stained. After ˜2 days, pigs may pass yellow fibrinonecrotic casts that have formed in the ileum. Most affected pigs recover spontaneously, but a significant number develop chronic necrotic enteritis with progressive emaciation. The hemorrhagic form is characterized by cutaneous pallor, weakness, and passage of hemorrhagic or black, tarry feces. Pregnant gilts may abort. Lesions may occur anywhere in the lower half of the small intestine, cecum, or colon but are most frequent and obvious in the ileum. The wall of the intestine is thickened, and the mesentery may be edematous. The mesenteric lymph nodes are enlarged. The intestinal mucosa appears thickened and rugose, may be covered with a brownish or yellow fibrinonecrotic membrane, and sometimes has petechial hemorrhages. Yellow necrotic casts may be found in the ileum or passing through the colon. Diffuse, complete mucosal necrosis in chronic cases causes the intestine to be rigid, resembling a garden hose. Proliferative mucosal lesions often are in the colon but are detected only by careful inspection at necropsy. In the profusely hemorrhagic form, there are red or black, tarry feces in the colon and clotted blood in the ileum. Altogether, L. intracellularis is a particularly great cause of losses in swine herds in Europe as well as in the United States.

L. intracellularis is an obligate, intracellular bacterium which cannot be cultured by normal bacteriological methods on conventional cell-free media and has been thought to require cells for growth. S. McOrist et al., Infection and Immunity, Vol. 61, No. 19, 4286-4292 (1993) and G. Lawson et al., J. of Clinical Microbiology, Vol. 31, No. 5, 1136-1142 (1993) discuss cultivation of L. intracellularis using IEC-18 rat intestinal epithelial cell monolayers in conventional tissue culture flasks. In U.S. Pat. Nos. 5,714,375 and 5,885,823, both of which patents are herein incorporated by reference in their entireties, cultivation of L. intracellularis in suspended host cells was described. Spirochaetal colitis is caused by the Brachyspira pilosicoli bacteria. This infection generally affects 10-20 week old growers/finishers. It is characterized by a non-fatal wasting diarrhea of growing pigs that results in an increased number of days needed to finish. The diarrhea also results in reduction in feed efficiency and produces watery diarrhea or loose stools. About half of the pigs may show transient to persistent, to watery to mucoid green to brownish diarrhea, without blood. The clinical signs are more common 10-14 days after mixing and changing of the feed. Swine dysentery is caused by the bacteria Brachyspira hyodysentheriae. There are twelve known sero-types at this time. Clinical signs in established herd include diarrhea, a rapid loss of condition in some pigs, a hairy appearance, dehydration, painful abdomen, and the death of one or two pigs before other pigs show any signs. In a key outbreak in naïve herds, all age groups from suckling piglets to adult sows can be effected.

Porcine Epidemic Diarrhea (PED) is caused by a coronavirus somewhat similar to that which causes TGE. This virus is widespread in Europe. The virus damages the villi in the gut thus reducing the absorptive surface, with attendant loss of fluid and dehydration. After introduction of the virus into a susceptible breeding herd, a strong immunity develops over two to three weeks. The colostral immunity then protects the piglets. The virus usually disappears spontaneously from breeding herds particularly small ones (<300 sows). Acute outbreaks of diarrhea occur when the virus is first introduced into a susceptible population. In such cases up to 100% of sows may be affected, showing a mild to very watery diarrhea. Two clinical pictures are recognized: PED Type I only affects growing pigs whereas PED Type II affects all ages including sucking pigs and mature sows. The incubation period is approximately 2 days and diarrhea lasts for 7 to 14 days. In sucking pigs the disease can be mild or severe with mortalities up to 40%. In large breeding herds, particularly if kept extensively, not all the females may become infected the first time around and there may be recrudescence. This only occurs in piglets suckling from sows with no maternal antibodies and is therefore sporadic.

Clostridium is a ubiquitous gram-positive bacteria, of the family clostridiaceae, usually found in the soil, but which also occurs naturally in the gut of most animals. C. difficile infections in swine are characterized by severe mesocolonic edema, diarrhea, and edema in other tissues such as the hydrothorax. Clostridium enteritis in swine is caused by C. perfringens, and is characterized by chronic enteritis, which is accompanied by diarrhea, weight loss and fever. Infection with C perfringens types A, B and C causes severe enteritis, dysentery, toxemia, and high mortality in young calves. Types B and C both produce the highly necrotizing and lethal β toxin that is responsible for the severe intestinal damage. This toxin is sensitive to proteolytic enzymes, and disease is associated with inhibition of proteolysis in the intestine. Sow colostrum, which contains a trypsin inhibitor, has been suggested as a factor in the susceptibility of young piglets. The disease can cause sudden death in piglets less than one week old, and is most common within 3 days of birth. In older piglets, Clostridium enteritis causes a thickening of the small intestine making absorption of food and nutrients difficult. Piglets usually die as a result of a combination of the infection and lack of nutrients. Death may occur in a few hours, but less severe cases survive for a few days, and recovery over a period of several days is possible. Hemorrhagic enteritis with ulceration of the mucosa is the major lesion in all species. Grossly, the affected portion of the intestine is deep blue-purple and appears at first glance to be an infarction associated with mesenteric torsion. Smears of intestinal contents can be examined for large numbers of gram-positive, rod-shaped bacteria, and filtrates made for detection of toxin and subsequent identification by neutralization with specific antiserum. Clostridium novyi has been suspected but not yet confirmed as a cause of sudden death in cattle and pigs fed high-level grain diets, and in which pre-existing lesions of the liver were not detectable. The lethal and necrotizing toxins (primarily α toxin) damage hepatic parenchyma, thereby permitting the bacteria to multiply and produce a lethal amount of toxin. Usually, death is sudden with no well-defined signs. Affected animals tend to lag behind the herd, assume sternal recumbency, and die within a few hours. Most cases occur in the summer and early fall when liver fluke infection is at its height. The disease is most prevalent in 1- to 4-yr-old sheep and is limited to animals infected with liver flukes. Differentiation from acute fascioliasis may be difficult, but peracute deaths of animals that show typical lesions on necropsy should arouse suspicion of infectious necrotic hepatitis. The most characteristic lesions are the grayish yellow necrotic foci in the liver that often follow the migratory tracks of the young flukes. Other common findings are an enlarged pericardial sac filled with straw-colored fluid, and excess fluid in the peritoneal and thoracic cavities. Usually, there is extensive rupture of the capillaries in the subcutaneous tissue, which causes the adjacent skin to turn black (hence the common name, black disease). Clostridium septicum is found in soil and intestinal contents of animals (including man) throughout the world. Infection ordinarily occurs through contamination of wounds containing devitalized tissue, soil, or some other tissue-debilitant. Wounds caused by accident, castration, docking, insanitary vaccination, and parturition may become infected. General signs, such as anorexia, intoxication, and high fever, as well as local lesions, develop within a few hours to a few days after predisposing injury. The local lesions are soft swellings that pit on pressure and extend rapidly because of the formation of large quantities of exudate that infiltrates the subcutaneous and intramuscular connective tissue of the affected areas. Accumulations of gas are uncommon. Malignant edema associated with lacerations is characterized by marked edema, severe toxemia, and death in 24-48 hr. Tetanus toxemia is caused by a specific neurotoxin produced by Clostridium tetani in necrotic tissue. Almost all mammals, including swine, are susceptible to this disease. Although tetanus is worldwide in distribution, there are some areas, such as the northern Rocky Mountain section of the USA, where the organism is rarely found in the soil and where tetanus is almost unknown. In general, the occurrence of C tetani in the soil and the incidence of tetanus in man is higher in the warmer parts of the various continents. Clostridium tetani, an anaerobe with terminal, spherical spores, is found in soil and intestinal tracts. In most cases, it is introduced into the tissues through wounds, particularly deep puncture wounds, that provide a suitable anaerobic environment.

Escherichia coli is a bacteria of the enterbacteriaceae family and is one of the main types of bacteria naturally occurring in the small intestines of all mammals. Although usually harmless, some E coli strains can produce a number of exo- and endotoxins that cause infection and disease. Heat-labile (LT) and heat-stable (ST) exotoxins are actively produced by some strains and are responsible for causing scour. Shigela-like toxin type II variant (SLT-IIe), Stx2e and verotoxin edema disease act on the wall of the small arteries resulting in oedema. Endotoxins, such as Lipid A, play a role in mastitis and urinary tract infections. E. coli infection is characterized by a number of different symptoms depending on the particular strain involved, including diarrhea, sunken eyes, unthriftiness, visible weight loss, stunted growth, depression, bowel edema, mastitis, cystitis, pyelonephritis and death. E. coli can be classified and coded by their cell wall (O antigens) and fimbriae (F antigens). For example, scour is often associated with E. coli Abbotstown: O147, F4, F5, whereas bowel edema is associated with F18 fimbriae. Correctly identifying the code is essential to the selection of the correct vaccine. E. coli infections compromise a pig's immune system and deaths are often the result of secondary infections and disease.

Porcine circovirus is a small (17-22 nm in diameter), icosahedral, non-enveloped DNA virus, which contains a single-stranded circular genome. Porcine circovirus type 2 PCV2 shares approximately 80% sequence identity with porcine circovirus type 1 (PCV1). However, in contrast with PCV1, which is generally non-virulent, swine infected with PCV2 exhibit a syndrome commonly referred to as Post-weaning Multisystemic Wasting Syndrome (PMWS). PMWS is clinically characterized by wasting, paleness of the skin, unthriftiness, respiratory distress, diarrhea, icterus, and jaundice. In some affected swine, a combination of all symptoms will be apparent while other swine will only have one or two of these symptoms. During necropsy, microscopic and macroscopic lesions also appear on multiple tissues and organs, with lymphoid organs being the most common site for lesions. A strong correlation has been observed between the amount of PCV2 nucleic acid or antigen and the severity of microscopic lymphoid lesions. Mortality rates for swine infected with PCV2 can approach 80%.

Rotavirus infection is a virus infection that is widespread in pig populations. It is present in most if not all pig herds with virtually a 100% sero-conversion in adult stock. A further epidemiological feature is its persistence outside the pig where it is resistant to environmental changes and many disinfectants. Maternal antibodies persist for 3-6 weeks after which pigs become susceptible to infection but exposure does not necessarily result in disease. It is estimated that only 10-15% of diarrheas in pigs are initiated by a primary rotavirus infection. In a mature herd disease appears after piglets are 7 to 10 days of age. It becomes progressively less important with age. However if pathogenic strains of E. coli are present, severe disease can occur with heavy mortality.

Infection with Salmonella spp can produce diarrhea in animals of all ages, especially those that are stressed, closely stocked, or exposed to a heavily contaminated feed or water supply. Salmonellosis is caused by many species of salmonellae and characterized clinically by one or more of three major syndromes—septicemia, acute enteritis, and chronic enteritis. The incidence has increased with the intensification of livestock production. Although various types of Salmonella can cause infections in pigs, the classic salmonellas found in swine are S. choleraesuis and S. typhimurium. Their resulting clinical patterns of most salmonella are not distinct and different species of salmonellae tend to differ in their epidemiology. Plasmid profile and drug-resistance patterns are sometimes useful markers for epidemiologic studies. Septicemic salmonellosis is often associated with S choleraesuis. Infected piglets demonstrate a reluctance to move, anorexia, a high fever of 40.5 C-41.6 C, and may have a shallow cough. Piglets may also be found dead with cyanotic extremities. S choleraesuis is one of the rare diseases that can cause both pneumonia and diarrhea and mortality of infected piglets is often high. Enterocolitis is generally associated with the more common S typhimurium. Infections are characterized by yellow or watery diarrhea that may contain blood or mucus as the infection progresses. Mortality is low and often associated with dehydration and potassium deficiency from the diarrhea. Feces of infected animals can contaminate feed and water, fresh and processed meats from abattoirs, plant and animal products used as fertilizers or feedstuffs, pasture and rangeland, and many inert materials. S choleraesuis is rarely found in feed. It can also be passed directly from contact with an infected animal. Salmonella can survive for months in wet, warm areas such as in feeder pig barns or in water dugouts. Rodents and wild birds also are sources of infection. The prevalence of infection varies among species and countries and is much higher than the incidence of clinical disease, which is commonly precipitated by stressful situations such as sudden deprivation of feed, transportation, drought, crowding, parturition, and the administration of some drugs.

Transmissible gastroenteritis (TGE) is a disease of the intestines caused by a coronavirus (Transmissible gastroenteritis virus). It is in the same family as Porcine respiratory coronavirus, epidemic diarrhea virus, and Hemagglutinating encephalomyelitis virus. Initial clinical signs are watery diarrhea, vomiting, and anorexia. Piglets less than 21 days of age generally die, weaners become unthrifty, while growers, finishers, and adults are generally mildly affected and will survive if provided with adequate water. Effective salmonella vaccines are already available. One preferred vaccine is Enterisol® SC-54 (Boehringer Ingelheim Vetmedica, Inc., St. Joseph, Mo.). Such vaccines are preferably administered to an animal in need thereof prior to salmonella and/or PCV2 infection. Such vaccination can occur prior to, after, or be accompanied with vaccination against PCV2 infection. Conventional vaccination protocols can be followed for both salmonella and PCV2.

The term “immunogenic composition” as used herein, refers to any pharmaceutical composition containing a PCV2 and/or other antigen, which composition can be used to prevent or treat an enteric disease associated disease or condition in a subject. A preferred immunogenic composition can induce, stimulate or enhance the immune response against enteric diseases caused by PCV2 as well as PCV2 and any other enteric pathogen. The term thus encompasses both subunit immunogenic compositions, as described below, as well as compositions containing whole killed, or attenuated and/or inactivated PCV2 and/or any other enteric pathogen.

The term “subunit immunogenic composition” as used herein, refers to a composition containing at least one immunogenic polypeptide or antigen, but not all antigens, derived from or homologous to an antigen from PCV2. Such a composition is substantially free of intact PCV2. Thus, a “subunit immunogenic composition” is prepared from at least partially purified or fractionated (preferably substantially purified) immunogenic polypeptides from PCV2, or recombinant analogs thereof. A subunit immunogenic composition can comprise the subunit antigen or antigens of interest substantially free of other antigens or polypeptides from PCV2, or in fractionated form. A preferred PCV2 immunogenic subunit composition comprises the PCV2 ORF2 protein as described below, and in particular, any one of the PCV2 ORF2 proteins described in WO 06/072065.

The immunogenic composition as used herein most preferably comprises the polypeptide, or a fragment thereof, expressed by ORF-2 of PCV2. PCV2 ORF-2 DNA and protein used herein for the preparation of the compositions and within the processes provided in WO 06/072065, is a highly conserved domain within PCV2 isolates and thereby, any PCV2 ORF-2 would be effective as the source of the PCV ORF-2 DNA and/or polypeptide as used herein. A preferred PCV2 ORF-2 protein is that of SEQ ID NO: 11 of WO06/072065. A further preferred PCV2 ORF-2 polypeptide is provided as SEQ ID NO: 5 of WO06/072065 and a still further preferred PCV2 ORF-2 polypeptide or protein is provided as SEQ ID NO: 6 of WO06/072065. However, it is understood by those of skill in the art that this sequence could vary by as much as 6-10% in sequence homology and still retain the antigenic characteristics that render it useful in immunogenic compositions. The antigenic characteristics of an immunological composition can be, for example, estimated by the challenge experiment as provided by Example 4 of WO06/072065. Moreover, the antigenic characteristic of a modified antigen is still retained, when the modified antigen confers at least 70%, preferably 80%, more preferably 90% of the protective immunity as compared to the PCV2 ORF-2 protein, encoded by the polynucleotide sequence of SEQ ID NO:3 or SEQ ID NO:4 as provided in WO06/072065.

Preferably, said PCV2 antigen is
i) a polypeptide comprising the sequence of SEQ ID NO: 5, SEQ ID NO: 6, SEQ ID NO: 9, SEQ ID NO: 10 or SEQ ID NO: 11 of WO06/07065;
ii) any polypeptide that is at least 80% homologous to the polypeptide of i),
iii) any immunogenic portion of the polypeptides of i) and/or ii)
iv) the immunogenic portion of 11i), comprising at least 10 contiguous amino acids included in the sequences of SEQ ID NO: 5, SEQ ID NO: 6, SEQ ID NO: 9, SEQ ID NO: 10 or SEQ ID NO: 11 of WO06/072065,
v) a polypeptide that is encoded by a DNA comprising the sequence of SEQ ID NO: 3 or SEQ ID NO: 4 of WO06/072065.
vi) any polypeptide that is encoded by a polynucleotide that is at least 80% homologous to the polynucleotide of v),
vii) any immunogenic portion of the polypeptides encoded by the polynucleotide of v) and/or vi)
viii) the immunogenic portion of vii), wherein polynucleotide coding for said immunogenic portion comprises at least 30 contiguous nucleotides included in the sequences of SEQ ID NO: 3, or SEQ ID NO: 4 of WO06/072065.

Preferably any of those immunogenic portions have the immunogenic characteristics of PCV2 ORF-2 protein that is encoded by the sequence of SEQ ID NO: 3 or SEQ ID NO: 4 of WO06/07065.

An “immunological or immune response” to a composition or vaccine is the development in the host of a cellular and/or antibody-mediated immune response to the composition or vaccine of interest. Usually, an “immune response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in number or severity of, or lack of one or more of the symptoms associated with PCV2 infections as described above.

The terms “immunogenic” protein or polypeptide or “antigen”, as used herein, generally refer to an amino acid sequence which elicits an immunological response as described above. An “immunogenic” protein or polypeptide, as used herein, includes the full-length sequence of any PCV2 proteins, analogs thereof, or immunogenic fragments thereof. The term “immunogenic fragment” refers to a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

Synthetic antigens are also included within the definition, as for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998.

In a preferred embodiment of the present invention, an immunogenic composition that induces an immune response and, more preferably, confers protective immunity against the clinical signs of enteric disease, is provided.

The term “reduction of the incidence of and/or severity of enteric disease clinical signs”, as used herein, means the reduction of any apparent clinical symptoms normally associated with enteric diseases that allow a precise and undoubtful identification of enteric disease infection by its typical clinical appearance. “Reduction of incidence of enteric disease”, as used herein, also means that the number of animals which are affected by enteric disease, or at least one of the above mentioned clinical symptoms of enteric disease in a group of animals treated with the immunogenic composition provided herein, preferably with PCV2 antigen, alone or in combination with antigen from at least one other enteric disease causing organism, is lower as compared to the number of animals affected by enteric disease in a group of animals which are not treated with said PCV2 antigen, alone or in combination with the other enteric disease antigen. In this context, the term “lower” means a reduction of at least 5%, preferably at least 10%, more preferably at least 15%, even more preferably at least 20%, even more preferably at least 25%, even more preferably at least 30%, even more preferably at least 40%, and most preferably at least 50%. The tem “reduction of severity of enteric disease” as used herein, means, that the duration or severity of the clinically apparent phase of enteric disease is shortened, preferably with regard to one or more of the above mentioned symptoms of enteric disease. In this context, the term “is shortened” means that the average duration of the apparent clinical phase of the enteric disease, preferably with regard to one or more of the above mentioned symptoms of enteric disease, in a group of animals treated with the immunogenic composition provided herein, preferably with PCV2 antigen, alone or in combination with an antigen from another enteric disease causing organism, is shortened by at least 5%, preferably at least 10%, more preferably at least 15%, even more preferably at least 20%, even more preferably at least 25%, even more preferably at least 30%, even more preferably at least 40%, and most preferably at least 50%, as compared to a group of animals which are not treated with said PCV2 antigen, alone or in combination with said antigen from another enteric disease causing organism.

Those of skill in the art will understand that the composition used herein may incorporate known injectable, physiologically acceptable sterile solutions. For preparing a ready-to-use solution for parenteral injection or infusion, aqueous isotonic solutions, such as e.g. saline or corresponding plasma protein solutions, are readily available. In addition, the immunogenic and vaccine compositions of the present invention can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with allyl sucrose or with allyl pentaerythritol. Among them, there may be mentioned Carbopol 974P, 934P and 971P.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314, or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the composition can include one or more pharmaceutical-acceptable carriers. As used herein, “a pharmaceutical-acceptable carrier” includes any and all solvents, dispersion media, coatings, stabilizing agents, diluents, preservatives, antibacterial and antifungal agents, isotonic agents, adsorption delaying agents, and the like.

The immunogenic compositions can further include one or more other immunomodulatory agents such as, e.g., interleukins, interferons, or other cytokines. The immunogenic compositions can also include Gentamicin and Merthiolate. While the amounts and concentrations of adjuvants and additives useful in the context of the present invention can readily be determined by the skilled artisan, the present invention contemplates compositions comprising from about 50 μg to about 2000 μg of adjuvant and preferably about 250 μg/ml dose of the vaccine composition. Thus, the immunogenic composition as used herein also refers to a composition that comprises from about 1 ug/ml to about 60 μg/ml of antibiotics, and more preferably less than about 30 μg/ml of antibiotics.

The composition according to the invention may be applied intradermally, intratracheally, intravaginally, intramuscularly, or intranasally. In an animal body, it can prove advantageous to apply the pharmaceutical compositions as described above via an intravenous or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullary, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months and in different dosages. Vaccines or immunogenic compositions against the other enteric disease causing pathogens are preferably administered in their conventional manners.

Thus, one aspect of the invention provides for the use of an immunogenic composition for reducing or lessening the severity of clinical symptoms associated with enteric disease. Such a use generally comprises the step of administering a PCV2 antigen to a pig. In preferred forms, the antigen comprises ORF2 of PCV2, and more preferably comprises Circoflex (Boehringer Ingelheim Vetmedica, Inc., St. Joseph, Mo.). The administration of the antigen can be in any conventional form, including intradermal, intratracheal, intravaginal, intramuscular, intranasal, intravenous, intravascular, intraarterial, intraperitoneal, oral, intrathecal, subcutaneous, intracutaneous, intracardial, intralobal, intramedullar, or intrapulmonary, with intramuscular injectionadministration being preferred. In other preferred forms, an antigen from another enteric disease causing organism is also administered to the animal, although such administration does not have to be concurrent with the administration of the PCV2 antigen.

In another aspect of the present invention, a process for the production of a medicament for reducing or lessening the severity of clinical symptoms associated with enteric diseases provided. Generally, the process includes the steps of obtaining a PCV2 antigen, and combining said antigen with veterinary-acceptable carriers, pharmaceutical-acceptable carriers, or immunomodulatory agents, before administration to the animal. Again, in preferred forms, the PCV2 anitigen comprises ORF2 of PCV2, and more preferably Circoflex.

In yet another aspect of the present invention, a method of reducing the incidence of or lessening the severity of enteric disease is provided. Generally the method comprises the step of administering an effective amount of a PCV2 antigen to an animal, preferably a swine. Preferably, the administration is intradermal, intratracheal, intravaginal, intramuscular, intranasal, intravenous, intravascular, intraarterial, intraperitoneal, oral, intrathecal, subcutaneous, intracutaneous, intracardial, intralobal, intramedullar, or intrapulmonar. Preferably, the antigen comprises PCV2 ORF2, and more preferably Circoflex. Even more preferably, the administration occurs before exposure to or infection by PCV2. In some preferred forms, an effective amount of a second non-PCV2 antigen is also administered to the animal, although such administration does not have to be concurrent with the administration of the PCV2 antigen. Preferably, this other antigen is associated with enteric disease.

### BRIEF DESCRIPTION OF THE FIGURES

FIG. 1 is a graph illustrating the Average Daily Weight Gains by group and duration of time on test either 14 or 28 days due to necropsy on day 14 post-infection;

FIG. 2 is a graph representing gross lungs scores performed at necropsy. The chart represents the percentage of consolidation of the lung at the time of necropsy;

FIG. 3 is a graph representing Salmonella fecal processing, percent of positive samples in a treatment group during the study;

FIG. 4 is a graph illustrating results from tissue processing for the presence of Salmonella on days 14 and 28;

FIG. 5 is a graph illustrating results from PCR fecal testing for the presence of Lawsonia based on a percentage of samples per group; and

FIG. 6 is a graph illustrating tissue results for the presence of Lawsonia on both necropsy days 14 and 28 by PCR;

FIG. 7 is a graph showing overall IHC scores based on the treatment groups at necropsy;

FIG. 8 is a graph showing ELISA results for the presence of PCV2 in the serum; and

FIG. 9 is a graph illustrating PCR detection of PCV2 in feces.

### DETAILED DESCRIPTION OF THE PREFERRED EMBODIMENTS

The following example sets forth a preferred embodiment of the present invention. This example is illustrative in nature and nothing herein should be taken as a limitation of the overall scope of the invention.

### Example 1

### Materials and Methods

The 21 day study consisted of 6 treatment groups of 2-4 week old negative test pigs. Treatment group 1 (20 pigs) was designated as the “strict control” group and didn't receive a treatment. Treatment group 2 (20 pigs) was designated as “PCV2 and Salmonella” treated and received a Salmonella dose of 10 logs/mL accompanied with 5 logs/mL of PCV2. Treatment group 3 (20 pigs) was designated as “PCV2 and Lawsonia” treated and received a Lawsonia dose of 6.89 logs/mL and the PCV2 dose was 5 logs/mL. Treatment group 4 (20 pigs) was designated as “Lawsonia” treated and received a dose of 6.89 logs/mL. Treatment group 5 (20 pigs) was designated as “PCV2” treated and received 5 logs/mL. Treatment group 6 (20 pigs) was designated as “Salmonella” treated and received a dose of 10 logs/mL. Table 1 below summarizes the experimental design.

TABLE 1
Experimental Design

| Group | Number of Animals | Challenge |
|---|---|---|
| 1 | 20 | N/A |
| 2 | 20 | PCV2 and Salmonella |
| 3 | 20 | PCV2 and Lawsonia |
| 4 | 20 | Lawsonia |
| 5 | 20 | PCV2 |
| 6 | 20 | Salmonella |

On days 7, 14, 21 post-challenge, fecal and serum samples were collected and tested for Salmonella typhimurium, Lawsonia intracellularis, and PCV2. Fourteen days post-inoculation, 60 (half) of the animals were necropsied at random. Tissues and fecal samples were collected, and tested for the presence of Salmonella by selective enrichment and isolation. The serum collected was split into two vials for testing. The tissue and fecal material was also tested by Lawsonia PCR, PCV2-specific IHC, and quantitative PCR testing was also performed. On day 28, the remaining 60 animals were necropsied in the same manner. Feces, tissues and serum were collected and tested for Salmonella, Lawsonia, and PCV2.

Animals receiving PCV2 were given 5 logs₁₀ of material via stomach tube. Lawsonia challenge culture was tittered at 6.9 logs₁₀ and administered via stomach tube. The Salmonella was delivered orally and tittered at 10 logs₁₀. None of the animals had any complications from the challenge. Fecal samples were collected along with blood samples. Blood was centrifuged and serum was split into two samples for testing.

Serum and fecal samples were collected weekly. A gram of fecal material was tested upon arrival for Salmonella by enrichment and the remaining fecal material was frozen to be tested by PCR at a later date. The gram of feces was mixed with Buffered Peptone Water, at 37° C. overnight incubation, and then 1 ml of the peptone mixture was transferred into RV (rappartport-vassildlis) medium for another 37° C. overnight incubation. Lastly, 10 μl of the RV mixture was struck out on an XLD plate and incubated overnight in a plastic container to maintain moisture. The plates were read the next day for Salmonella growth. If plates were determined to have Salmonella, it was confirmed by testing a single colony on Urea, Kliger, and SIM media tubes.

Lawsonia testing consisted of thawing the fecal material and using a sterile disposable 10 μL loop to add a loopful of material to an eppendorf tube. A Qiagen extraction procedure was performed followed by PCR with Lawsonia-specific primers. Tissues were processed in the same fashion, collecting a small amount of tissue and adding it to an eppendorf tube for Qiagen extraction.

### Results and Discussion

### Clinical Scores and Testing

The animals remained healthy throughout the study. There were a few elevated temperatures recorded and most of the animals maintained temperatures between 103° F. to 104° F. A select number of animals ran a fever of 105° F. throughout the study, but only one of the animals exhibited a high temperature for more than one consecutive day and it was an animal in the Salmonella only group that exhibited an elevated temperature for 5 consecutive days (ID # 290). There were a total of three animals that had elevated temperatures prior to challenge. The majority came after challenge with a total of 23 elevated temperatures in the no challenge group, 23 elevated in the PCV/Salmonella group, 34 in the PCV/Lawsonia group, 12 in both the Lawsonia only and PCV only groups, and 15 in the Salmonella only group.

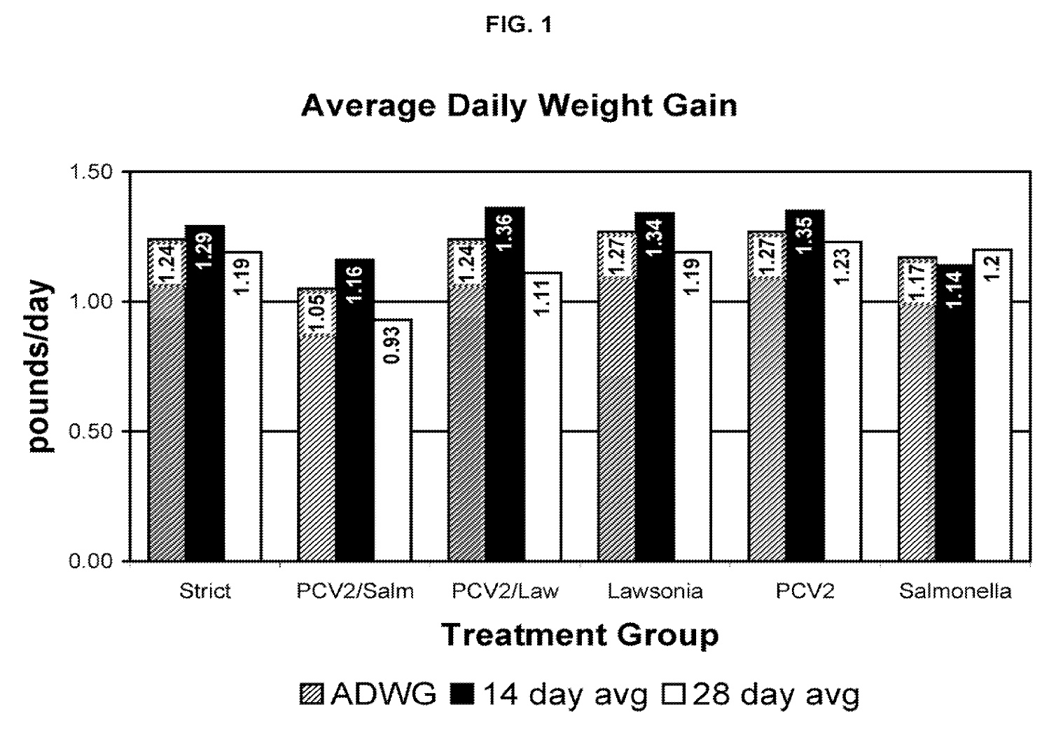

As seen in FIG. 1, the highest average daily weight gain was in the Lawsonia only and PCV2 only groups with an average daily weight gain of 1.27 pounds a day; followed by the strict controls and the PCV2/Lawsonia combination groups each gaining an average of 1.24 pounds a day. The lowest gains were in the groups receiving Salmonella and the PCV2/Salmonella group with 1.17 and 1.05 pounds per day, respectively. The figure below shows average weight gain for the respective number of days the animals were in the study (14 or 28 days).

### Lung Lesions

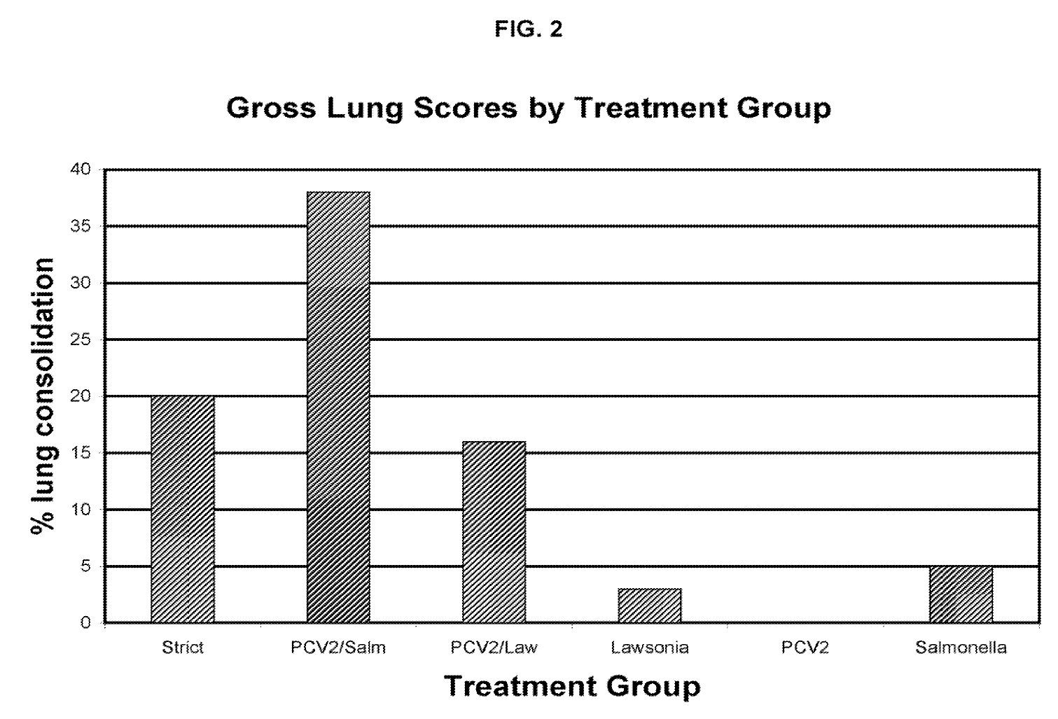

The strict controls had 20% lung consolidation. Salmonella alone had 5% lesions, yet when it was co-infected with PCV2, the lesions were enhanced to 38% lung consolidation. The PCV2/Lawsonia combination was also more severe than each pathogen individually. The PCV2/Lawsonia combination had 16.4% consolidation, PCV2 had 0% and Lawsonia had 3.4% lung consolidation, both were better then the strict control animals, as can be seen in FIG. 2.

### Salmonella Isolation by Fecal Processing

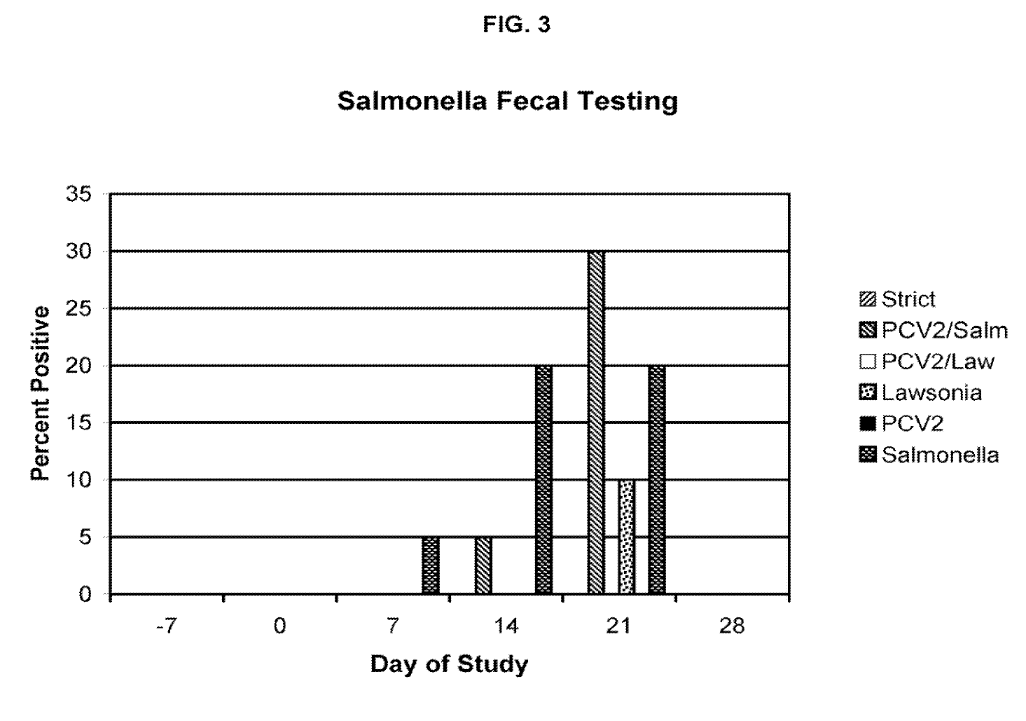

The Salmonella treated groups had the highest percent of positive isolation results. Still, the isolation was low in regards to the number of fecal samples that were positive after the enrichment process. FIG. 3 summarizes the Salmonella fecal isolation testing. Strict controls remained negative throughout the study. The Lawsonia group did have 10% recovery on day 21, then no recovery for any group on day 28.

### Salmonella Tissue Processing for Isolation

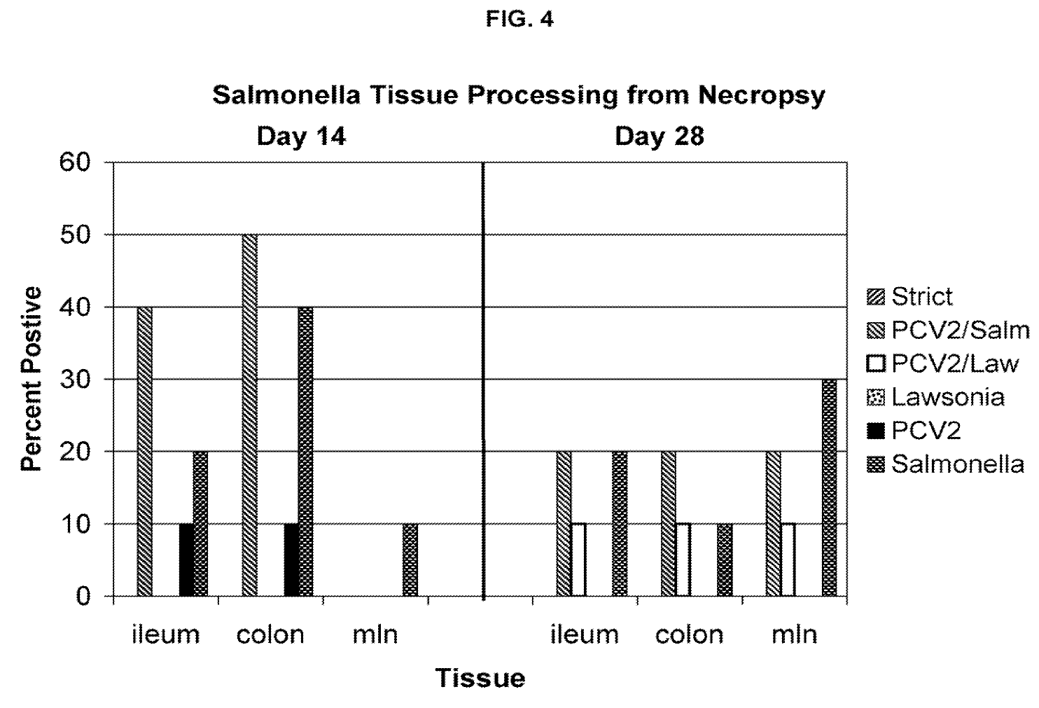

Overall the tissues had a numerically higher percent of positive samples in comparison to the fecal samples. The treatment groups challenged with Salmonella had the most positive tissues. There was at least one positive tissue from each Salmonella treated group at necropsy. Except on day 14, mesenteric lymph nodes from the combination group of PCV2 and Salmonella, the animals received a challenge of Salmonella, but it was not sub cultured out of the mesenteric lymph node. There was also some Salmonella isolation from the PCV2 only group. On day 28, there was a higher percentage of positive tissues in the PCV2/Lawsonia group, group that did not receive a Salmonella challenge yet, Salmonella was recovered from the tissues. FIG. 4 summarizes the results of Salmonella tissue testing.

### Lawsonia Fecal PCR (Qualitative)

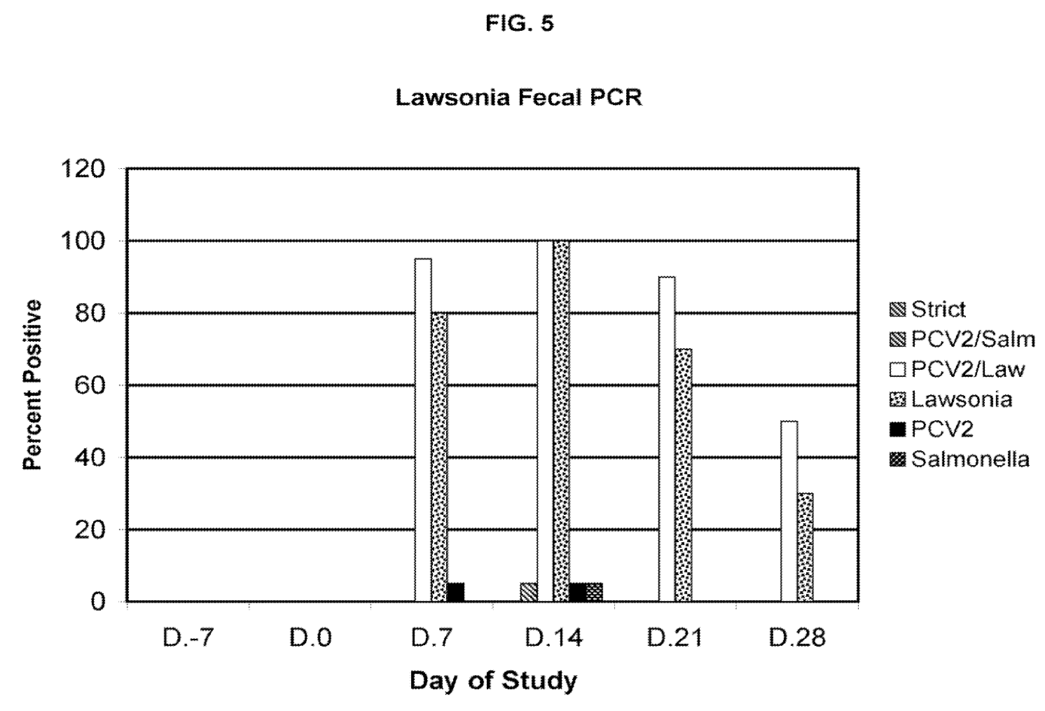

The PCV2 Treatment group 5, PCV2 only group, had one animal positive for Lawsonia on day 7 and 14, ID #293 and #273, respectively. The Salmonella and PCV2/Salmonella groups each had one animal positive on Day 14. Besides those four animals, the only groups that generated positive animals by feces were the animals that received a challenge dose of Lawsonia. See FIG. 5 for Lawsonia fecal testing. The Lawsonia challenged groups were 60% or higher in the number of positive samples one week after challenge, but on day 28 the percentage of positive fecal samples began slowly dropping.

### Lawsonia and Tissue PCR (Qualitative)

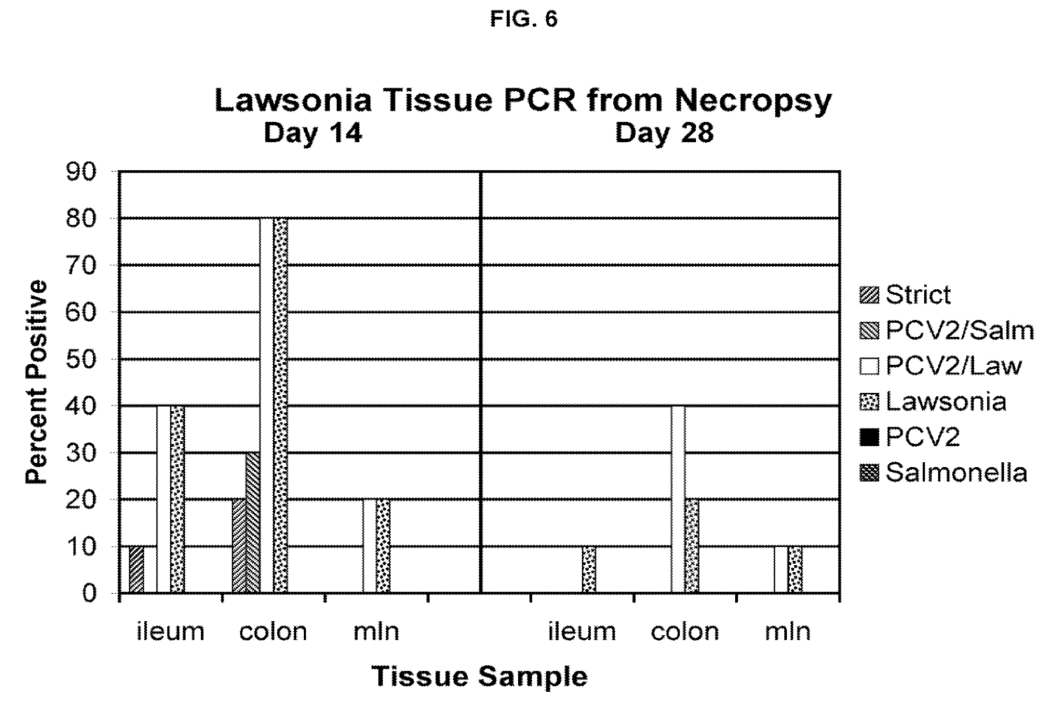

As presented in FIG. 6, Tissue PCR from the necropsy on day 14 was positive for Lawsonia in 4 out of the 6 groups in the colon. The Salmonella only and PCV2 only groups did not have any positive tissues on day 14 or 28. Day 28 had fewer positive tissues than day 14 tissues, with the groups that had been challenged with Lawsonia exhibiting positive tissues on day 28. There were also tissues that tested positive on day 14 for the presence of Lawsonia in the strict controls.

### Lawsonia Fluorescent Antibody (FA) Testing

The only groups to respond in the Lawsonia based Fluorescent Antibody test were groups that received a challenge exposure of Lawsonia. The Lawsonia group reached a maximum of 40% positive on day 21. The combo group of PCV2/Lawsonia reached a maximum seroconversion of 60% on day 28 (see Table 2 for Lawsonia FA scores).

TABLE 2
The percentage of positive samples based on Lawsonia
Fluorescent Antibody testing.

| Treatment Groups | D.-7 | D.0 | D.7 | D.14 | D.21 | D.28 |
|---|---|---|---|---|---|---|
| Strict (group 1) | 0% | 0% | 0% | 0% | 0% | 0% |
| PCV2/Salm (group | 0% | 0% | 0% | 0% | 0% | 0% |
| 2) | | | | | | |
| PCV2/Law (group 3) | 0% | 0% | 0% | 0% | 20% | 60% |
| Lawsonia (group 4) | 0% | 0% | 0% | 0% | 40% | 30% |
| PCV2 (group 5) | 0% | 0% | 0% | 0% | 0% | 0% |
| Salmonella (group 6) | 0% | 0% | 0% | 0% | 0% | 0% |

### Tissue IHC (ImmunoHistoChemistry)

PCV2-specific IHC results on the small intestine had moderate scores in all groups on day 14. Day 28, the strict controls were negative in the small intestine while the other five groups had animals that scored positive for PCV2 IHC small intestine. In regards to the large intestine on day 14 all groups had a positive score for PCV2-specific. On day 28, Lawsonia only and Salmonella only treatment groups had moderate scores while the other four treatment groups were negative by IHC.

Lawsonia-specific IHC results for small and large intestine showed all groups contained positive animals on day 14, but on day 28 all treatment groups were considered negative. Lastly, Salmonella-specific IHC showed all groups contained a few positive animals on day 14. On day 28, the Lawsonia only group had one positive animal.

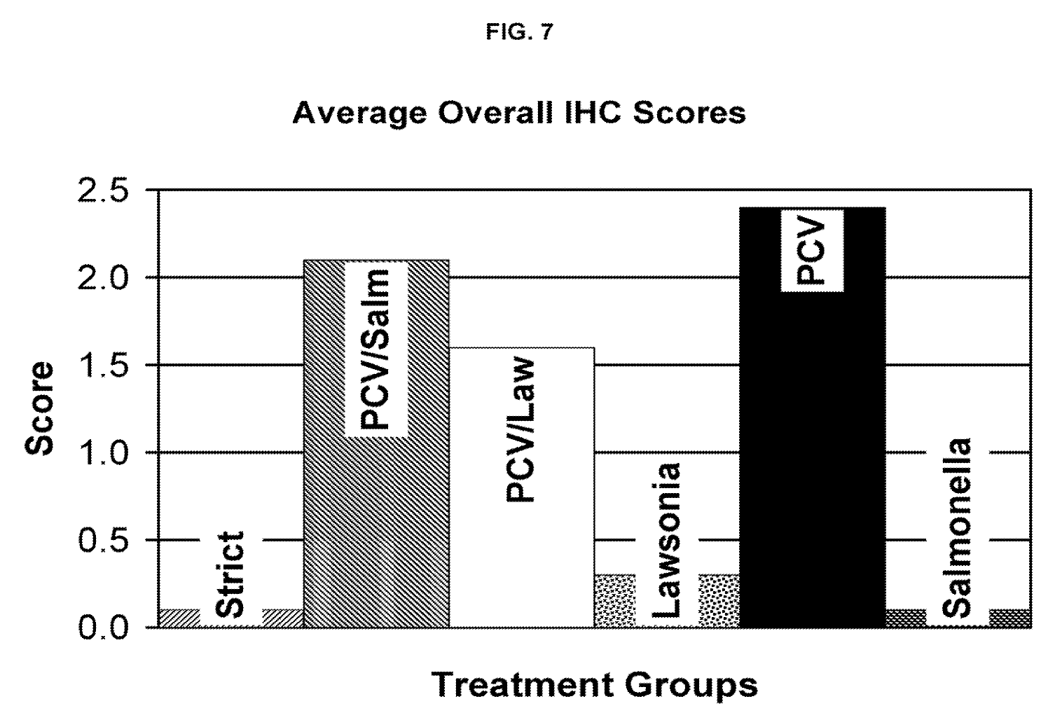

As presented in FIG. 7, the overall the IHC scores were low to moderate in the amount of virus/bacteria present on each slide. The highest overall score (IHC, enteric lesions, and lymphoid lesions) was in the PCV2 treatment group with an IHC score of 2.4. It should be noted that the treatment groups with the highest scores were the groups treated with PCV2, while the Strict controls, Lawsonia, and the Salmonella treatment groups had the least amount of bacteria/virus present at the time of slide scoring.

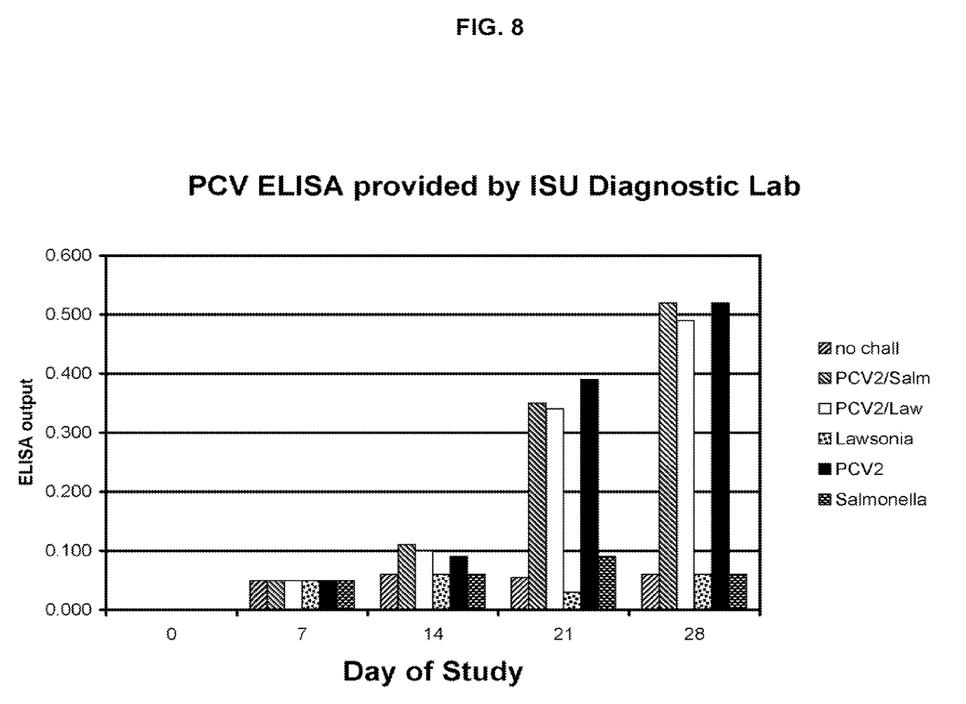

The PCV2 ELISA, graphic results presented in FIG. 8, is interpreted such that an S/P (samples/percentage of positive samples) ratio of 0.2 and above is considered PCV2 positive. Based on that interpretation, the three treatment groups that were challenged with PCV2 tested positive by ELISA. The other three treatment groups did not have any animals that tested positive by the PCV2 ELISA. Seroconversion to PCV2 did not occur until somewhere between the period of 14-21 days post-challenge.

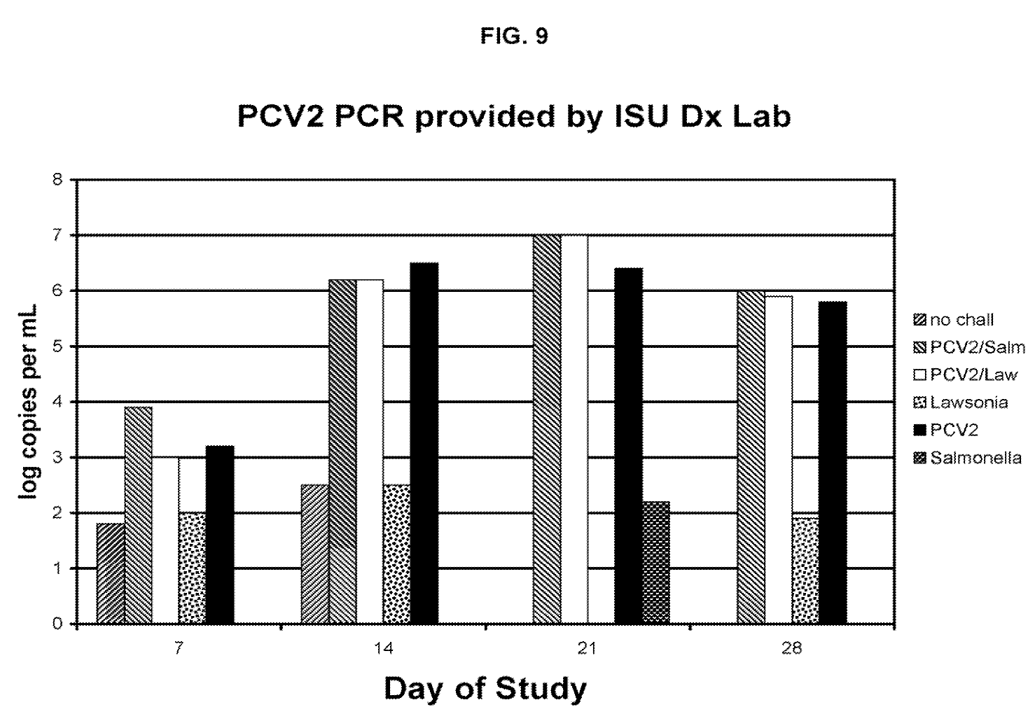

FIG. 9 shows that PCV2 was detected on all key sampling dates in the treatment groups that received a PCV2 challenge. It should be noted that on day 14, 4 animals from non-PCV2 related groups tested positive. This included the strict control group, animals 297 and 300, and from the Lawsonia only group, animals 283 and 284. On day 21, one animal was reported positive from a non-PCV2 treated group; Salmonella-only group animal no. 239.

### DISCUSSION

This study was designed to investigate the relationship of age at time of challenge with the severity of disease. The animals were randomly assigned to a treatment group with a random number generator.

The results showed an increase in enteric disease symptoms when PCV2 infection was concurrent with infection of an enteric pathogen. FIG. 1 shows that average daily weight gain was lower in the groups infected with PCV2 and Lawsonia or PCV2 and Salmonella, with the lowest average daily weight gain being in the PCV2/Salmonella group. The PCV2/Salmonella group exhibited 33% more lung lesions (38%) than were exhibited in the group infected with Salmonella alone (5%). Lung lesions were increased by 13% in the PCV2/Lawsonia group, with animals infected with Lawsonia alone exhibiting 3.4% lung lesions compared to 16.4% lung lesions in the PCV2/Lawsonia group.

Fecal testing showed that the PCV2/Salmonella group had the highest positive fecal testing and the PCV2/Lawsonia group exhibited positive fecal testing, while the strict controls remained negative throughout the study. Tissue PCR revealed that from the 14 day necropsy, there were Lawsonia positive tissues in 4 out of the 6 groups in the colon. Fluorescent antibody testing showed that the combination PCV2/Lawsonia group exhibited significantly higher seroconverion than those infected with Lawsonia alone.

Overall, it was concluded that clinical symptoms of enteric disease were increased in quantity and quality when the enteric pathogen was in combination with PCV2. As described above, in almost every category of testing, positive results for enteric disease were heightened in those groups infected with PCV2 and an enteric pathogen, when compared to groups only infected with the enteric pathogen. PCV2 infection increases the severity of enteric disease in animals, particularly when in combination with an enteric pathogen, such as Lawsonia or Salmonella, leading to the conclusion that PCV2 vaccination in animals prone to enteric disease will lessen the severity and frequency of symptoms associated with enteric disease infection.

We claim:

1. A method of inducing an immunogenic response against an enteric pathogen comprising the step of:
administering a composition comprised of a PCV2 antigen and a pharmaceutically acceptable carrier or adjuvant to an animal.

2. The method of claim 1, wherein said composition is administered by injection.

3. The method of claim 1, wherein said composition additionally comprises an antigen from an enteric pathogen.

4. The method of claim 3, wherein said enteric pathogen is E. coli.

5. The method of claim 3, wherein said enteric pathogen is S. typhimurium.

6. The method of claim 3, wherein said enteric pathogen is L. intracellularis.

7. The method of claim 3, wherein said enteric pathogen is selected from the group consisting of: Lawsonia intracellularis, Salmonella, S. typhimurium, S. choleraesuis, Astroviruses, Rotavirus, Transmissible gastroenteritis virus, Brachyspira spp, B. hyodysenteriae, B. pilosicoli, Clostridium, C. difficile, C. perfringens, C. novyi, C. septicum, C. tetani, Porcine enteric picornaviruses, Porcine enteric caliciviruses, coronavirus, E. coli and combinations thereof.

8. A method of lessening the incidence of enteric clinical signs of an enteric pathogen comprising the step of:
administering a composition comprised of a PCV2 antigen and a pharmaceutically acceptable carrier or adjuvant to an animal.

9. The method of claim 8, wherein said composition is administered by injection.

10. The method of claim 8, wherein said composition additionally comprises an antigen from an enteric pathogen.

11. The method of claim 10, wherein said enteric pathogen is E. coli.

12. The method of claim 10, wherein said enteric pathogen is S. typhimurium

13. The method of claim 10, wherein said enteric pathogen is L. intracellularis.

14. The method of claim 10, wherein said enteric pathogen is selected from the group consisting of: Lawsonia intracellularis, Salmonella, S. typhimurium, S. choleraesuis, Astroviruses, Rotavirus, Transmissible gastroenteritis virus, Brachyspira spp, B. hyodysenteriae, B. pilosicoli, Clostridium, C. difficile, C. perfringens, C. novyi, C. septicum, C. tetani, Porcine enteric picornaviruses, Porcine enteric caliciviruses, coronavirus, E. coli, and combinations thereof.

15. A method of reducing the severity of clinical signs of enteric infection comprising the step of:
administering a composition comprising a PCV2 antigen and a pharmaceutically acceptable carrier or adjuvant to an animal.

16. The method of claim 15, wherein said composition is administered by injection.

17. The method of claim 15, wherein said composition additionally comprises an antigen from an enteric pathogen.

18. The method of claim 17, wherein said enteric pathogen is E. coli.

19. The method of claim 17, wherein said enteric pathogen is S. typhimurium

20. The method of claim 17, wherein said enteric pathogen is L. intracellularis.

21. The method of claim 16, wherein said enteric pathogen is selected from the group consisting of: Lawsonia intracellularis, Salmonella, S. typhimurium, S. choleraesuis, Astroviruses, Rotavirus, Transmissible gastroenteritis virus, Brachyspira spp, B. hyodysenteriae, B. pilosicoli, Clostridium, C. difficile, C. perfringens, C. novyi, C. septicum, C. tetani, Porcine enteric picornaviruses, Porcine enteric caliciviruses, coronavirus, E. coli and combinations thereof.
